(19) United States
(12) Patent Application Publication
(10) Pub. No.: US 2005/0032199 A1
(43) Pub. Date: Feb. 10, 2005

(54) Cell culture apparatus

(75) Inventor: Seiya Takahashi, Saitama-shi (JP)

Correspondence Address: SCULLY SCOTT MURPHY & PRESSER, PC, 400 GARDEN CITY PLAZA, GARDEN CITY, NY, 11530, US

(73) Assignee: OLYMPUS CORPORATION, TOKYO (JP)

(21) Appl. No.: 10/844,077

(22) Filed: May 12, 2004

(30) Foreign Application Priority Data
May 16, 2003 (JP) 2003-138737

(51) Int. Cl. C12M 1/36; C12M 1/00

(52) U.S. Cl. 435/286.5; 435/303.1

(57) ABSTRACT

To discharge liquid samples such as reagent, and incubate cells, while preventing the evaporation of the culture solution and eliminating the burden placed on cells, there is provided a cell culture apparatus omprising: a board which holds cell-containing droplet culture solution, a holding chamber which accommodates the board in an interior space set to an incubation temperature, and a droplet supplying device which supplies droplet mist at near incubation temperature into the interior space of the holding chamber.

### BACKGROUND OF THE INVENTION

1. Field of the Invention

This invention relates to a cell culture apparatus used for the incubation of cell-containing droplet culture solutions.

Priority is claimed on Japanese Patent Application No. 2003-138737, filed May 16, 2003, the content of which is incorporated herein by reference.

2. Description of the Related Art

Cell arrays are widely known and used for the purpose of incubating living cells. They consist of a board of several cm, and minute mediums arranged regularly in a high-density formation. Incubation of the cells occurs in these mediums. Cell arrays are used in drug screening, and allow evaluation of cell response towards drugs to be processed at high throughput. Many methods are known for this type of screening. One such method of screening uses micro-titer-like plates containing a virtual well formed by an arrangement of relatively hydrophilic regions (domain) and relatively hydrophobic regions (for example, see Published Japanese Translation No. 2002-502955 of PCT International Publication (Paragraph No. 0011-0140, FIG. 5, FIG. 6).

The microtiter-like plates are built by attaching a covering top member onto a bottom member, while leaving minute gaps between the two members. Hydrophobic regions and hydrophilic regions are formed on the surface of the bottom member, and a plurality of hydrophilic regions are provided being surrounded by hydrophobic regions. These hydrophilic regions are independent of one another. Hence it is possible to hold different types of liquid samples simultaneously in a plurality of hydrophilic regions.

When using such microtiter-like plates for drug screening of cells, the cell-containing culture solution is firstly introduced onto the plates. At this point, the culture solution preferentially attaches to the hydrophilic regions of the virtual well. Then, the culture solution is removed from the plate, resulting in a state where the cell-containing culture solution is held in the hydrophilic regions. Subsequently, reagents are added to the cells exposing them to chemical compounds, the cells are dissolved after incubation, and the composition is analyzed to measure the effect of the drug.

### SUMMARY OF THE INVENTION

This invention takes such circumstances into consideration with an object of providing a cell culture apparatus which can discharge liquid samples such as reagent, and incubate cells, while preventing the evaporation of the culture solution and eliminating the burden placed on cells.

The present invention is to provide a cell culture apparatus including: a board which holds cell-containing droplet culture solution; a holding chamber which accommodates the board in an interior space set to an incubation temperature; and a droplet supplying device which supplies droplet mist at near incubation temperature into the interior space of the holding chamber.

### BRIEF DESCRIPTION OF THE DRAWINGS

FIG. 1 is a diagram showing a first embodiment of a cell culture apparatus according to the present invention.

FIG. 2 is a cross-section showing a cell array holding chamber and droplet supplying part of the cell culture apparatus shown in FIG. 1.

FIG. 3 is a cross-section showing a cell dispensation apparatus of the cell culture apparatus shown in FIG. 1.

FIG. 4 is a diagram showing a state of discharging cell-containing droplet culture solution onto the cell array board.

FIG. 5 is a cross-section showing a state where the passage part of the cell dispensation apparatus is inserted into the cell array holding chamber.

FIGS. 6A, and 6B are an enlarged cross-sections of the passage part of the cell dispensation apparatus, FIG. 6A being a diagram showing the state of the cylindrical piezoelectric element before voltage is applied, and FIG. 6B being a diagram showing the state of the cylindrical piezoelectric element when voltage is applied.

FIG. 7 is a diagram showing a drive voltage wave form applied to the cylindrical piezoelectric element.

FIG. 8 is a diagram showing a second embodiment of a cell culture apparatus according to the present invention.

FIG. 9 is a cross-section showing a cell array holding chamber of the cell culture apparatus shown in FIG. 8.

FIG. 10 is a cross-section showing a dispensation head unit of the cell culture apparatus shown in FIG. 8.

FIG. 11 is an enlarged view showing a state where droplet mist is present in the surroundings of a droplet culture solutions discharged onto the cell array board, due to the cell culture apparatus shown in FIG. 1.

FIG. 12 is a cross-section showing a modified example of the passage part of the cell dispensation apparatus and dispensation head unit.

FIG. 13 is a cross-section showing a modified example of the cell dispensation apparatus and dispensation head unit.

### DETAILED DESCRIPTION OF THE INVENTION

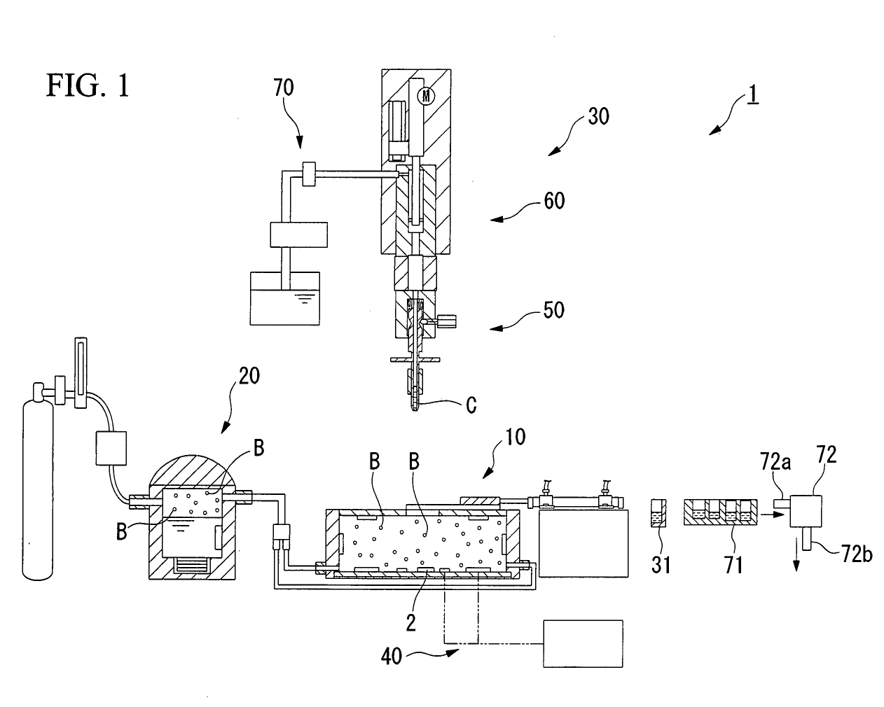

Hereunder is a description of a first embodiment of a cell culture apparatus 1 according to the present invention, with reference to FIG. 1 to FIG. 7.

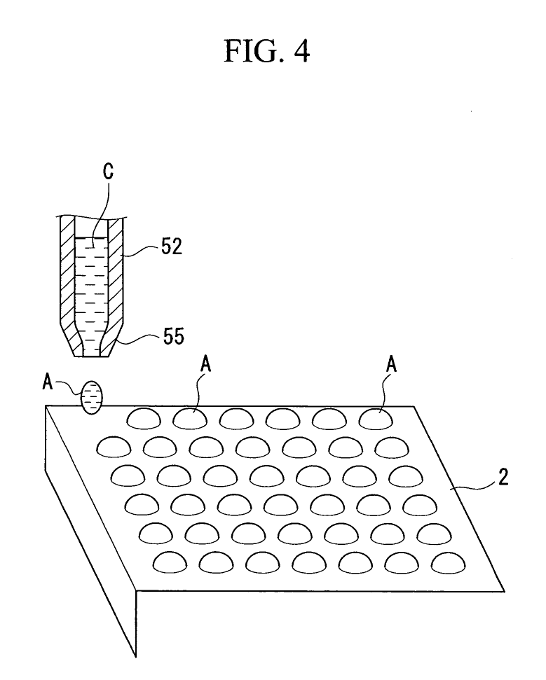

As shown in FIG. 1 and FIG. 4, the cell culture apparatus 1 of the present embodiment includes: a cell array board (board) 2 which holds a cell-containing droplet culture solution A; a cell array holding chamber (holding chamber) 10 which accommodates the cell array board 2 in its interior space set to incubation temperature; a droplet supplying part (droplet supplying device) 20 which supplies droplet mist B at near incubation temperature into the interior space of the cell array holding chamber 10; and a cell dispensation apparatus (liquid discharging device) 30 which discharges cell suspensions C, that is cell-containing culture solutions and liquid such as drugs towards the cell array board 2.

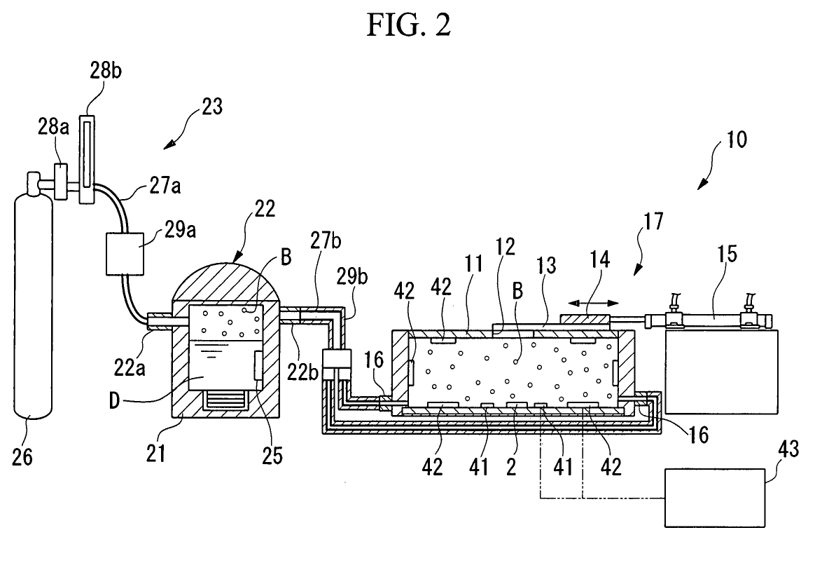

As shown in FIG. 2, the cell array board 2 is formed in a size of width 2 cm, depth 2 cm, and thickness 0.5 mm for example, making it possible to hold a plurality of droplet culture solutions A on its upper surface. This cell array board 2 is accommodated in the cell array holding chamber 10.

The cell array holding chamber 10 is formed in a box shape having a lid 11 of width 10 cm, depth 7 cm, and height 5 cm, including a temperature holding mechanism (temperature holding device) 40 which maintains its interior at a near incubation temperature of 37° C.±0.5° C.

The temperature holding mechanism 40 has a temperature sensor 41 arranged near the cell array board 2 held inside the cell array holding chamber 10, heaters 42 which heat the cell array board or in the vicinity, and a temperature control unit (controlling device) 43 which controls the output of the heater 42 based on the detected values of the temperature sensor 41.

The temperature sensor 41 is a thermocouple for example, and a plurality of these are arranged surrounding the cell array board 2, as shown in FIG. 2. A plurality of heaters 42 are arranged on the lower surface, side surface and upper surface inside the cell array holding chamber 10. Similarly to the heaters 42, a plurality of temperature sensors 41 may be arranged inside the cell array holding chamber 10.

In the lid 11 of the cell array holding chamber 10, an opening 12 is formed for inserting a passage part 52 (described later) of the cell dispensation apparatus 30 mentioned above. A pair of linear guides 13 are provided on both sides of the opening 12. On these linear guides 13, a pair of shutters 14 which are formed in a size to cover the opening 12 are moveably arranged. On one side of the shutters 14, an air cylinder 15 is connected, and the shutters 14 are moved (left and right in the plane of the page) by the air cylinder 15 making it possible to open and close the opening 12. Therefore, the linear guides 13, shutters 14 and air cylinder 15 constitute the opening and closing device 17 which opens and closes the opening 12.

The shutters 14 do not completely fill the opening 12, but are set to leave a small gap even in the closed state. The opening 12 is formed to be positioned directly above the cell array board 2.

The cell array holding chamber 10 has on both side walls a spray inlet 16 for droplet mist B supplied by the droplet supplying part 20 to be introduced inside. The bottom surface of the cell array holding chamber 10 is formed by transparent glass or other transparent material, so that the cell array board 2 can be observed from the lower side by an inverted microscope (not shown in the drawing).

The droplet supplying part 20 has a humidifier container (liquid holding container) 21 which holds deionized water (liquid) D, an ultrasonic humidifier (droplet generating device) 22 which applies ultrasonic vibration to the deionized water D held in the humidifier container 21 to generate the droplet mist B, and an air flow generation part (air flow generating device) 23 which generates air flow to send the droplet mist B generated in the humidifier container 21 into the cell array holding chamber 10.

The ultrasonic humidifier 22 is formed in a box shape with a lid covering the humidifier container 21. On the bottom of the humidifier container 21 is provided an oscillator 24 capable of ultrasonic vibration. The ultrasonic humidifier 22 thereby has a function to transfer the vibration of the oscillator 24 to the deionized water D and then generate the droplet mist B from the surface of the water.

The ultrasonic humidifier 22 has a thermostatic heater (liquid temperature adjusting device) 25 which adjusts the temperature of the deionized water D held in the humidifier container 21. That is, the thermostatic heater 25 adjust the temperature of the deionized water D at near incubation temperature of 37° C.±0.5° C. Due to this the droplet mist B generated by this deionized water D is adjusted to near the incubation temperature of 37° C.±0.5° C.

Furthermore, in the ultrasonic humidifier 22, an air inlet 22a to introduce air flow sent from the air flow generation part 23, and an air outlet 22b to exhaust this air flow are provided.

The generation efficiency of droplet mist B can be improved by controlling the water level of the deionized water D stored in the humidifier container 21 to a level close to the convergence point of the ultrasonic waves.

The air flow generation part 23 has a compressed gas cylinder 26 filled with air with a carbon dioxide concentration of 5% at a compression of 150 kgf/cm², and is connected to the air inlet 22a of the ultrasonic humidifier 22 through a pipe 27a. The air outlet 22b of the ultrasonic humidifier 22 is connected to the spray inlets 16 of the cell array holding chamber 10 through a pipe 27b. The pipe 27b branches out into two pipes and connects to both spray inlets 16. Therefore, air (gas) in the compressed gas cylinder 26 is introduced into the cell array holding chamber 10 while containing the droplet mist B generated in the ultrasonic humidifier 22.

The airflow generation part 23 includes a pressure adjustment regulator 28a which adjusts the pressure of the air flow generated by the compressed gas cylinder 26, a flow rate adjustment valve (flow rate adjusting device) 28b which adjusts the flow rate, and a pipe air conditioner (gas temperature adjusting device) 29a which adjusts the temperature of the air sent into the cell array holding chamber 10. That is, the pipe 27a has, in order from the exit side of the compressed gas cylinder 26, the pressure adjustment regulator 28a, the flow rate adjustment valve 28b and the pipe air conditioner 29a. Accordingly, it is possible to send the air in the compressed gas cylinder 26, that not only has an adjusted pressure and flow rate, but also an air temperature at near incubation temperature of 37° C.±0.5° C. into the ultrasonic humidifier 22. Furthermore, the pipe 27b has attached therearound a silicon rubber heater 29b which warms the pipe 27b up to a near incubation temperature of 37° C.±0.5° C. That is, the silicon heater 29b, similarly to the pipe air conditioner 29a, is a part of the gas temperature adjusting device which adjusts the temperature of the air sent into the cell array holding chamber 10.

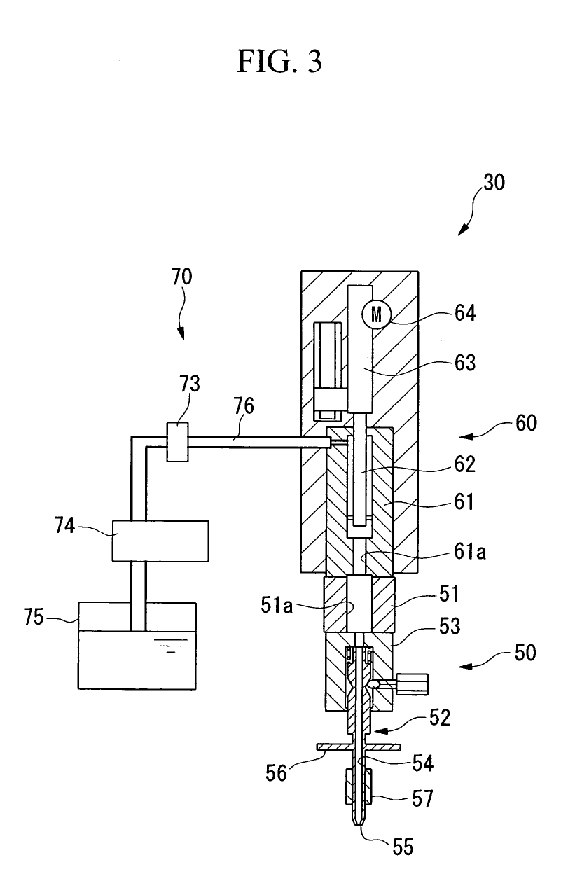

The cell dispensation apparatus 30, as shown in FIG. 3, includes a discharging head part 50 and a syringe pump part 60, and is moveable in XYZ directions by a transport device (not shown in the figure). Due to this, it is possible to move between a cell suspension storage container 31, a microtiter plate 71 (described later), and the cell array holding chamber 10, and to all positions of the cell array holding chamber 10, as shown in FIG. 1. Furthermore, the cell suspension storage container 31 has a thermostatic heater (not shown in the figure) and adjusts a cell suspension C inside the suspension storage container at a near incubation temperature of 37° C.±0.5° C.

The discharging head part 50, as shown in FIG. 3, has a cylindrical piezoelectric element 51 and a passage part 52. This cylindrical piezoelectric element 51 is fixed on one side of the syringe block 61 of the syringe pump part 60. The side opposite to the cylindrical piezoelectric element 51 has a passage part 52 which is detachably fixed through a joint member 53. Furthermore, the center of the cylindrical piezoelectric element 51 is a cavity 51a, and electrodes are formed on the inner circumferential face and outer circumferential face. The electrodes are divided into an inner circumferential electrode as the negative electrode (ground) and an outer circumferential electrode as the positive electrode, so that a voltage can be applied from a driving circuit (not shown) to the respective electrodes. Due to this, when the voltage is applied to the cylindrical piezoelectric element 51, it can expand and contract in the axial direction, and the passage part 52 is also movable in the axial direction according to the movement of the cylindrical piezoelectric element 51.

The passage part 52 has a passage 54 thereinside, as well as a nozzle 55 with a of diameter 75 μm at its tip. This passage 54 is connected to the cavity 51a of the cylindrical piezoelectric element 51 through the passage of the joint member 53.

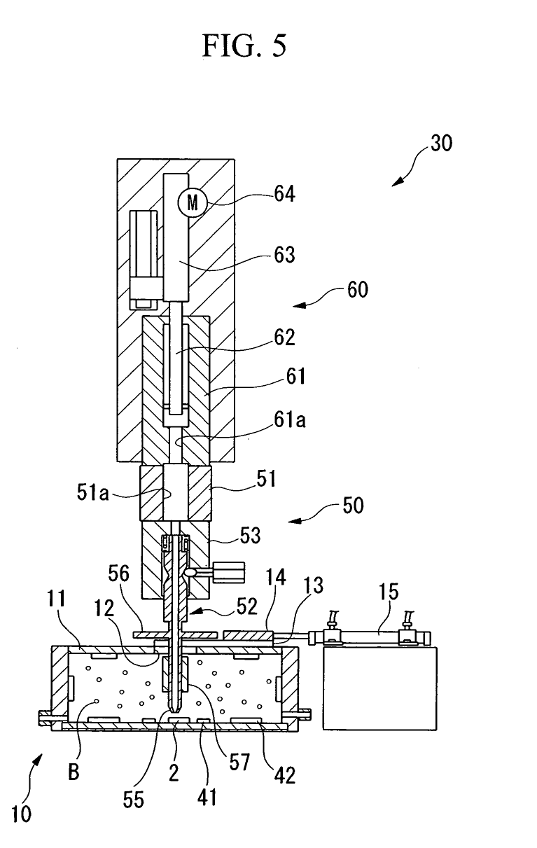

The passage part 52 has a flange part (shielding device) 56 which closes the gap between the opening 12 of the cell array holding chamber 10 and the cell dispensation apparatus 30, as shown in FIG. 5. That is, the flange part 56 is formed on the outer circumference of the passage part 52 to close the gap of the opening 12 when the passage part 52 is inserted into the cell array holding chamber 10. The flange part 56 is formed in the position where the distance from the flange part 56 to the nozzle 55 is approximately equal to that from the bottom surface of the cell array holding chamber 10 to the lid 11. The diameter of the flange part 56 is approximately twice that of the opening 12.

Furthermore, on the outer circumference of the tip of the passage part 52, a cylindrical heater 57 is provided to warm the passage part 52 up to a near incubation temperature of 37° C.±0.5° C. together with the air conditioner (not shown).

The syringe pump part 60, as shown in FIG. 3, includes in addition to the aforementioned syringe block 61, a piston 62, a rack and pinion mechanism 63 and a motor 64. The syringe block 61 has a passage 61a thereinside, and connects to the cavity 5la inside the cylindrical piezoelectric element 51. Hence the syringe block 61 connects to the nozzle 55 through the respective passages. The piston 62 is arranged inside this passage 61a, and one end of the piston 62 is connected to the motor 64 through the rack and pinion mechanism 63. The motor 64 is driven thereby so that the piston 62 is moveable reciprocatingly along the passage 61a (up and down in the plane of the page).

The cell dispensation apparatus 30, as shown in FIG. 1 and FIG. 3, includes a drug dispensation apparatus 70. This drug dispensation apparatus 70 has a microtiter plate 71 which holds a plurality of drugs, a wash tank 72 to wash the inside and outside of the cell dispensation apparatus 30, an electromagnetic valve 73, a water pump 74 and a water tank 75. The electromagnetic valve 73, water pump 74 and water tank 75 are provided on the syringe pump part 60.

That is to say, the passage 61a of the syringe block 61 has the water tank 75 connected to it through a pipe 76, and on this pipe 76 in order from the water tank 75, is the water pump 74 and the electromagnetic valve 73. By driving the water pump 74 after the electromagnetic valve 73 is operated in the opening state, water from the water tank 75 can be introduced to the passage 61a of the syringe block 61, making it possible to wash the passage of the cell dispensation apparatus 30.

In the wash tank 72, washing liquid is supplied from an inlet 72a from a washing liquid transporting device (not shown), and the washing liquid supplied is exhausted from outlet 72b. That is, a flow of washing liquid is formed from the inlet 72a to the outlet 72b inside the wash tank 72. Therefore, it is possible to wash the outer circumferential face of the passage part 52 by moving the cell dispensation apparatus 30 by the transporting device, and immersing the passage part 52 in the wash tank 72.

Hereunder is a description of a case where cells are incubated in the cell culture apparatus 1 configured in this manner.

Firstly, before the cell array board 2 is made to hold the cell-containing culture solution A, the cell culture apparatus 1 is set to an initial state. That is to say, as shown in FIG. 1 to FIG. 3, the temperature of the cell array holding chamber 10 and the cell array board 2 is adjusted to a near incubation temperature of 37° C.±0.5° C. by the temperature holding mechanism 40. That is, the temperature control unit 43 controls the temperature of the respective heaters 42 based on the detected values from the temperature sensor 41, to the desired near incubation temperature.

Moreover, the thermostatic heater 25 in the ultrasonic humidifier 22 is operated, presetting the temperature of the deionized water D inside the humidifier container 21 to a near incubation temperature of 37° C.±0.5° C. Furthermore, similarly to the thermostatic heater 25, the pipe air conditioner 29a, the silicon rubber heater 29b, the cylindrical heater 57 and the cell suspension storage container 31 are previously warmed up to a near incubation temperature of 37° C.±0.5° C.

Next, air from the compressed gas cylinder 26 is adjusted by the pressure adjustment regulator 28a and the flow rate adjustment valve 28b to a pressure range of 0.05 kgf/cm² to 0.20 kgf/cm² and a flow rate range of 0.1 l/min to 1.0 l/min, and supplied to the ultrasonic humidifier 22. At the same time as this air is being supplied, the oscillator 24 of the ultrasonic humidifier 22 is made to produce ultrasonic vibrations, generating the droplet mist B inside the humidifier container 21. The particle size of the droplet mist B generated is determined by the ultrasound frequency, and has a diameter of 2 μm to 10 μm for example in the present embodiment.

The droplet mist B generated inside the humidifier container 21 is transported by the air supplied by the compressed gas cylinder 26 and introduced into the cell array holding chamber 10 from the two spray inlets 16 through the pipe 27b. At this point the droplet mist B and air have been adjusted to a near incubation temperature of 37° C.±0.5° C. by the thermostatic heater 25, the pipe air conditioner 29a and the silicon rubber heater 29b. The droplet mist B is continuously supplied to the cell array holding chamber 10 by the air of the compressed gas cylinder 26. At this point, part of the droplet mist B flowed in is emitted together with the air from the small gap between the opening 12 and the shutter 14 to the outside, preventing an increase in pressure in the cell array holding chamber 10. Therefore, the inside of the cell array chamber 10 is filled with the droplet mist B maintaining a near incubation temperature of 37° C.±0.5° C.

After the initial state mentioned above is obtained, the cell dispensation apparatus 30 is moved by the transporting device, and the passage part 52 is immersed in the cell suspension storage container 31. After immersion, the motor 64 is driven to move the piston 62, sucking up about 5 μl of the cell suspension C through the nozzle 55 into the passage 54. At this time, since the passage 54 is warmed up by the cylindrical heater 57, the temperature of the cell suspension C sucked in is maintained at a near incubation temperature of 37° C.±0.5° C.

After the cell suspension C is sucked in, the transporting device moves the cell dispensation apparatus 30 to directly above the shutters 14 of the cell array holding chamber 10, that is the opening 12. Then, the shutters 14 are moved by the air cylinder 15 to open the opening 12, and the cell dispensation apparatus 30 is lowered by the transporting device so that the passage part 52 is inserted into the cell array holding chamber 10. At this point, the passage part 52 is inserted until the distance between the lower surface of the flange part 56 and the upper surface of the lid 11 of the cell array holding chamber 10 is about 1 mm, as shown in FIG. 5. In this state, the distance between the nozzle 55 at the tip of passage part 52 and the upper surface of the cell array board 2 is about 0.5 mm.

Next, the cell suspension C is discharged onto the cell array board 2 as the droplet culture solution A from the nozzle 55.

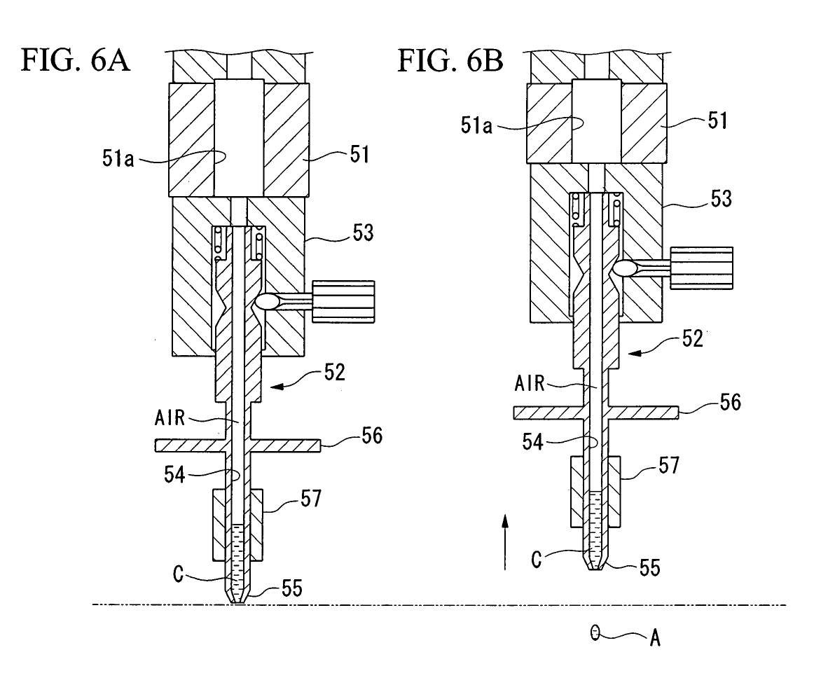

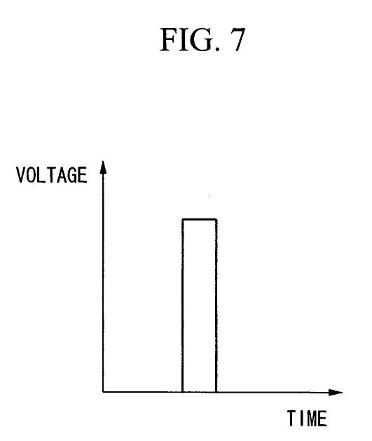

As shown in FIG. 6A, after the cell suspension C is sucked up, the passage part 54 is in a state where the cell suspension C exists at the tip and air exists above it. Here, as shown in FIG. 7, a positive pulse voltage is applied to both electrodes of the cylindrical piezoelectric element 51 by the driving circuit. When the voltage is applied, the cylindrical piezoelectric element 51 contracts along the axial direction (upwards in the plane of the page), as shown in FIG. 6B. With the movement of the cylindrical piezoelectric element 51, the passage part 52 also moves simultaneously. At this point, the cell suspension C held in the passage 54 of the passage part 52 is unable to follow the rapid movement of the passage part 52, and an inertia force acts on it in a direction opposite to the passage part 52 movement, that is towards the cell array board 2. As a result, the pressure at the tip of the passage 54 is increased so that the cell suspension C is discharged from the nozzle 55 in droplet form. That is, the cell-containing droplet form culture solution A is discharged.

The movement of the cylindrical piezoelectric element 51 is a small amount of around 2 μm, and the change in pressure inside the passage 54 corresponding to the movement of the cylindrical piezoelectric element 51 is absorbed by the expansion and compression of air. The amount of the droplet culture solution A discharged depends on the physical properties of the liquid sample, the driving voltage, and the number of cells. In this embodiment, a rectangular wave of 40V and 100 μsec is applied to discharge the culture solution A of 0.1 nl tol 0.0 nl.

As mentioned above, after one droplet of the culture solution A is discharged, the cell dispensation apparatus 30 is moved a small distance in the XY direction (planar direction) by the transporting device while maintaining the distance between the nozzle 55 and cell array board 2 constant. After movement, the droplet culture solution A is discharged from the nozzle 55. In this way, the discharge of the culture solution A and small movements of the nozzle 55 are repeated so that the culture solution A is serially discharged and held on the cell array board 2, as shown in FIG. 4. The time required for all of the culture solution A to be discharged ranges from approximately 100 to 500 seconds. Meanwhile, the opening 12 of the cell array holding chamber 10 is closed by the flange part 56 leaving a small gap. Therefore a large amount of the droplet mist B can not be exhausted from inside the cell array holding chamber 10 to the outside. Finally, the cell dispensation apparatus 30 is raised by the transporting device, withdrawing it from the cell array holding chamber 10, and the shutters 14 are closed by operating the air cylinder 15.

On the other hand, regarding the droplet culture solution A held on the cell array board 2, the internal temperature of the cell array holding chamber 10 is maintained at near incubation temperature, resulting in evaporation of water from the surface, serving to decrease the droplet volume. However, the inside of the cell array holding chamber 10 is filled with the suspended droplet mist B which is evenly spread over a wide space due to the air from the compressed gas cylinder 26. Accordingly, the droplet mist B adheres culture solutions A and becomes absorbed by the culture solutions A, increasing the droplet volume of the culture solution A. Therefore, the amount of water lost in the culture solutions A due to evaporation forms an equilibrium state with the amount of water absorbed from droplet mist B, resulting in the culture solutions A being maintained at a constant droplet volume. Moreover, since the temperature of the droplet mist B has been adjusted to near incubation temperature, when it is absorbed by the culture solution A, it does not apply any burden on the cells due to temperature.

In the present embodiment, the culture solutions A on the cell array board 2 can be held for 3 hours or more by setting the flow rate of the droplet mist B within the range of 10 μl/min to 100 μl/min.

As mentioned above, after the droplet culture solution A has been discharged onto the cell array board 2 to form the cell culture mediums, the drug is added to the cell culture mediums. That is to say, first, the cell dispensation unit 30 is moved by the transporting device in order to immerse the passage part 52 in the wash tank 72. As a result, the outer circumferential face of the passage part 52 is washed. Moreover the inside of the passage 54 is washed by opening the electromagnetic valve 73, driving the water pump 74, and introducing washing water from the wash tank 75 into the passage 54. After washing, the passage part 52 is raised by the transporting device, and the cylinder 63 is moved by the motor 64 to suck for example 10 μl of air into the passage 54. After the air is sucked in, the passage part 52 is inserted into the desired drug among the drugs held on the microtiter plate 71, by the transporting device, and the cylinder 63 is moved to suck for example 5 μl of drug into the passage 54, similarly to when air was sucked.

Then, using the same procedure mentioned above for discharging the culture solution A onto the cell array board 2, the drug is added to the cell culture mediums, that is, the respective culture solutions A. When different types of drugs are added, for each drug, the outer circumferential face of the passage part 52 must be repeatedly washed in the wash tank 72, and after the inside of the passage 54 is washed by the washing water, the desired drug must be sucked from the microtitier plate 71 to add to the respective culture solutions A.

When addition of the drug is completed, incubation of the cells is performed. That is to say, since the cell array holding chamber 10 has air containing 5% carbon dioxide supplied to it at all times, and is adjusted to a near incubation temperature, incubation can be performed inside the cell array holding chamber 10 itself. Moreover, once incubation has been performed for a predetermined time, the cells on the cell array board 2 can be observed by a fluorescence microscope or the like, so that cell viability determination and cell activity level assay can be performed to determine the effect of the drug.

As mentioned above, this cell culture apparatus 1 includes inside its cell array holding chamber 10a droplet supplying part 20 which supplies droplet mist B at near incubation temperature. Therefore the cell-containing droplet culture solution A held on the cell array board 2 has surrounding it a presence of the droplet mist B. That is, in this state, it is easy for the droplet mist B to adhere to the culture solution A. Therefore, evaporation of the culture solution A can be prevented by absorbing water from the droplet mist B to compensate for the amount of water lost through evaporation. Furthermore, since the droplet mist B is maintained at a temperature near incubation temperature, when it adheres to the culture solution A, it will not place a burden on cells.

The droplet mist B is sent into the cell array holding chamber 10 by using air flow from the compressed gas cylinder 26 so that the droplet mist B can be reliably sent in mist form. As a result, after reaching the cell array holding chamber 10, the droplet mist B is scattered over a large space due to the air flow so that it becomes possible to evenly adhere to respective culture solutions A on the cell array board 2.

Moreover, since the droplet supplying part 20 includes the flow rate adjustment valve 28b which adjusts the flow rate of the air generated by the air flow generation part 23, it is possible to adjust the amount of air and also the amount of droplet mist B supplied to the cell array holding chamber 10. Therefore, an optimal amount of the droplet mist B can be supplied to the culture solutions A held on the cell array board 2 according to factors such as the number and size, so that evaporation of the culture solutions A can be prevented more reliably.

Furthermore, since it includes the pipe air conditioner 29a and the silicon rubber heater 29b which adjust the temperature of the air sent into the cell array holding chamber 10 from the compressed gas cylinder 26, it is possible to adjust the air temperature sent into the cell array holding chamber 10 at near incubation temperature. Therefore, the burden on the cells due to temperature change can be decreased.

Moreover, since the ultrasonic humidifier 22 includes the thermostatic heater 24 which adjusts the temperature of the deionized water D, this deionized water D can be warmed up to near incubation temperature so that droplet mist B at near incubation temperature can be reliably generated.

Furthermore, since the cell array holding chamber 10 includes the temperature holding mechanism 40, it is possible to maintain its interior at near incubation temperature. Therefore, the cell-containing culture solution A can be accommodated in an optimal environment, and the burden on cells due to temperature change can be decreased. Specifically, since the temperature control unit 43 adjusts the respective heaters 42 which heat the cell array board 2, or in the vicinity thereof, based on the detected values from the temperature sensor 41, it is possible to maintain the surroundings of the cell array board 2 at near incubation temperature with high accuracy.

Furthermore, since the cell dispensation apparatus 30 is provided, liquid such as drugs can be discharged as required to add to the culture solutions A. Moreover, since the cell array holding chamber 10 includes the opening and closing device 17 which opens and closes the opening 12, the cells can be accommodated in the holding chamber 10 which is maintained at the optimal environment while being isolated from the outside at all times, other than when drugs need to be added to the culture solutions A, and the burden on the cells due to temperature change and the possibility of evaporation can be decreased.

Additionally, since the cell dispensation apparatus 30 includes the flange part 56, a large amount of the droplet mist B can be prevented from being exhausted from the cell array holding chamber 10 to outside, even when discharging drugs to the culture solutions A.

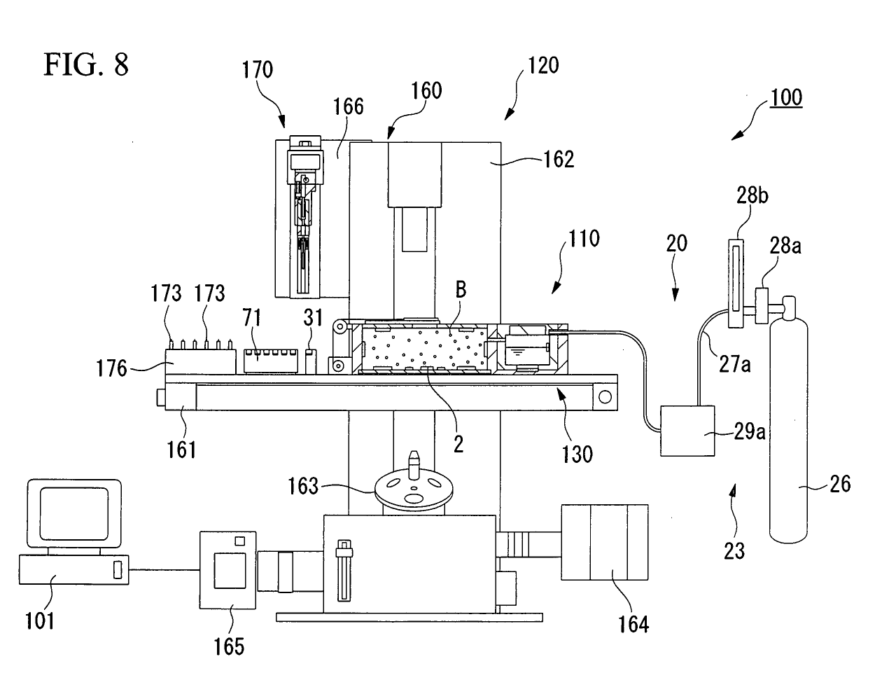

Next is a description of a second embodiment of the present invention, with a reference to FIG. 8 to FIG. 10. In the second embodiment, the same reference symbols denote the parts the same as components in the first embodiment, and their description is omitted.

The difference between the first embodiment and the second embodiment is that in the first embodiment, the droplet mist B was supplied irrespective of the liquid volume of the culture solution A accommodated inside the cell array holding chamber. However in the second embodiment the cell culture apparatus 100 detects the liquid volume of the culture solution A and adjusts the amount of droplet mist B supplied according to the detected value.

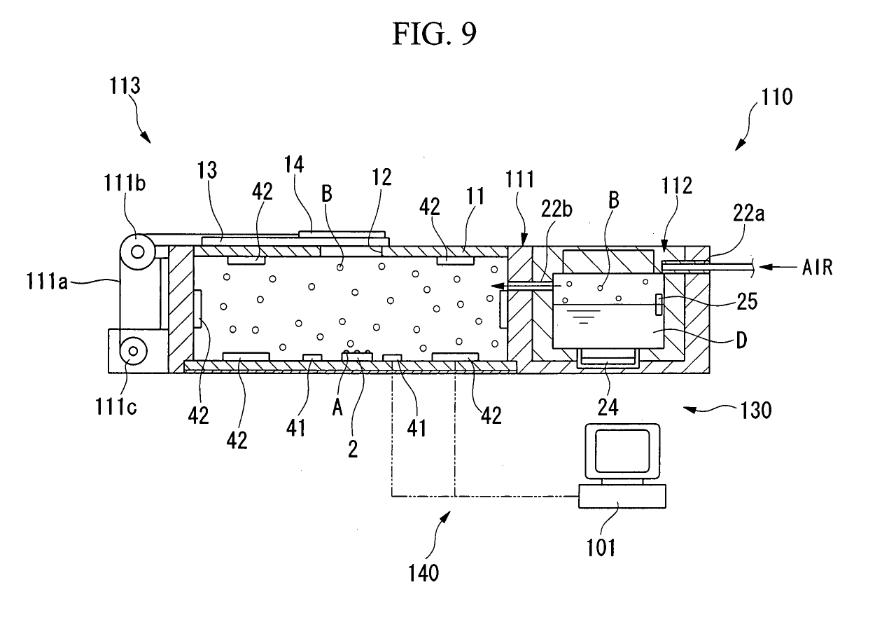

That is to say, the cell culture apparatus 100 of the present embodiment as shown in FIG. 8 and FIG. 9, includes a liquid volume detection mechanism (liquid volume detecting device) 120 which detects the liquid volume of the culture solution A held on the cell array board 2 in the cell array holding chamber (holding chamber) 110, and a liquid volume adjustment mechanism (liquid volume adjusting device) 130 which adjusts the liquid volume of the droplet mist B supplied by the droplet supplying part 20 based on the detected value from the liquid volume detection mechanism 120.

The cell array holding chamber 110 has an integrated configuration where a mainframe 111 is combined with an ultrasonic humidifier (droplet generating device) 112 as shown in FIG. 9. The mainframe 111 is formed into a box shape having a lid 11, and inside is capable of accommodating the cell array board 2 and also temperature sensors 41 and heaters 42 are provided. In the present embodiment, a personal computer (hereafter PC) 101 adjusts the output of the heaters 42 based on the detected values from the temperature sensors 41. That is, the temperature sensors 41, the heaters 42 and the PC 101 form the temperature holding mechanism (temperature holding device) 140 which maintains the temperature of the interior of the mainframe 111 at a near incubation temperature of 37° C.±0.5° C. The cell array board 2 of the present embodiment is a 1 mm thick transparent polypropylene board with a fibronectin coat over its surface so cells can more easily adhere.

In the mainframe 111, an opening 12 is formed to allow the dispensation head unit 170 to be inserted inside. This opening 12 has arranged on both sides, a pair of linear guides 13, and a shutter 14 is arranged movable over these linear guides 13. This shutter 14 is urged by a spring (not shown) so as to close the opening 12. The shutter 14 has on the opposite side to the spring, a wire 111a connected thereto, and this is connected to a rotary actuator 111c through a pulley 111b. That is, by operating the rotary actuator 111c, the shutter 14 is pulled by a force against to the spring force, so that it is possible to open the opening 12. Moreover, by stopping the rotary actuator 111c, the shutter 14 is pulled by the spring force so that it is possible to open the opening 12.

That is, the linear guide 13, the shutter 14, the spring, wire 111a, the pulley 111b and the rotary actuator 111c constitute an opening and closing device 113 which opens and closes the opening 12.

The lid 11 of the mainframe 111 and shutter 14 are formed by transparent material such as transparent plastic. The operation of the rotary actuator 111c is controlled by the PC 101.

The oscillator 24 of the ultrasonic humidifier 112 has a driving voltage applied to it through a driving circuit (not shown) which is connected to the PC 101 though an interface, enabling variable control. Hence, the amount of droplet mist B generated by the ultrasonic humidifier 112 can be controlled by the PC 101. That is, the ultrasonic humidifier 112 and PC 101 constitute the liquid volume adjustment mechanism 130 mentioned above.

Inside the ultrasonic humidifier 112, there is a thermostatic heater arranged which adjusts the temperature of the deionized water D to a near incubation temperature. The droplet mist B generated is supplied into the mainframe 111 through the air outlet 22b together with air sent from the air flow generation part 23 through the air inlet 22a.

The cell array holding chamber 110 mentioned above, is mounted on the XY stage 161 of an inverted microscope 160, as shown in FIG. 8. This inverted microscope 160 includes a frame 162 which supports the XY stage 161 movably (horizontally and vertically in the plane of the page), an object lens 163 to observe the cell array board 2 in the cell array holding chamber 110 from its lower side, a light source 164 which irradiates light for observation, and an imaging part 165 which takes pictures of the images observed by the object lens 163. The observation images taken by the imaging part 165 can be loaded onto and saved on the PC 101. Furthermore, the PC 101 has a function to control operation of the imaging part 165 and XY stage 161.

Hence, the inverted microscope 160 and the PC 101 constitute the liquid volume detection mechanism 120 to detect the liquid volume of the culture solution A as mentioned above. That is, the liquid volume detection mechanism 120 has a function to detect the liquid volume of the culture solution A by image processing which measures the diameter of the droplet of the culture solution A by the imaging part 165. The PC 101 variably controls the oscillator 24 of the ultrasonic humidifier 112 based on the detected value.

The frame 162 movably supports the ZY stage 166. The ZY stage 166 has provided on it, a dispensation head unit (liquid discharging device) 170 which discharges liquid such as the cell suspensions C and drugs onto the cell array board 2. That is, the dispensation head unit 170 is movable in the ZY direction (vertically in the plane of the page and out of the plane of the page).

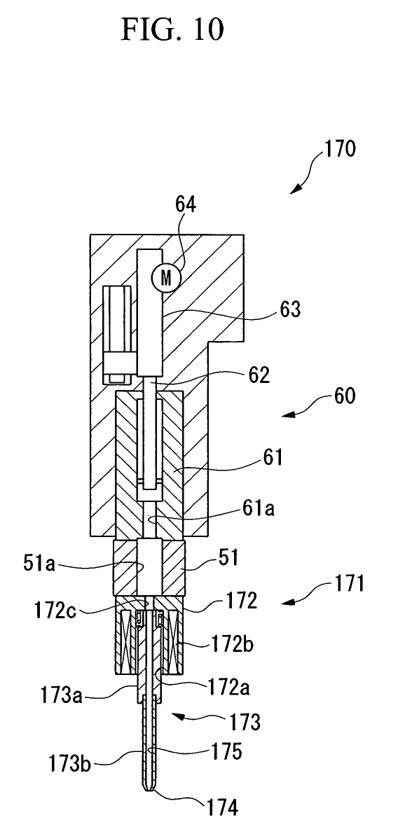

The dispensation head unit 170 includes a syringe pump part 60 and a discharge head part 171 as shown in FIG. 10.

The discharge head part 171 has a cylindrical piezoelectric element 51, a passage part detaching joint 172 and a passage part 173. The passage part detaching joint 172 is fixed on one end of the cylindrical piezoelectric element 51, and the passage part 173 is detachably fixed on the other end. The passage part detaching joint 172 has a passage part socket 172a which is a cavity in its interior, and an electromagnetic coil 172b arranged surrounding it. Electric current flows to the electromagnetic coil 172b from a circuit (not shown). That is, by using electromagnetic force, it is possible to detach the passage part 173. In the center of the passage part socket 172a, a passage 172c is formed and connected to the cavity 51a of the cylindrical piezoelectric element 51.

The passage part 173 has a rear part 173a and a front part 173b of different diameters. The rear part 173a is formed by a magnetic material such as SUS (stainless) 430 to have a diameter such that it can be inserted into the passage part socket 172a. The front part 173b is formed of ruby to have a smaller diameter than the rear part 173a, with a nozzle 174 of diameter 75 μm. In the passage part 173, a passage 175 is formed inside to connect between the nozzle 174 and the passage of the passage part detaching joint 172. The passage part 173 is inserted with an O-ring into the passage part socket 172a.

On the XY stage 161, along with the cell array holding chamber 110, a cell suspension storage container 31, a microtiter plate 71 and a passage part storage container 176 which stores a plurality of passage parts 173 for the dispensation head unit 170, are placed as shown in FIG. 8. Regarding this passage part storage container 176, similarly to the cell suspension storage container 31 and microtitier plate 71, it is possible to warm the plurality of passage parts 173 up to near incubation temperature by thermostatic heaters (not shown) or the like.

Hereunder is a description of the case where cells are incubated by the cell culture apparatus 100.

First, the inside of the mainframe 111 of the cell array holding chamber 110 is filled with the droplet mist B which is at a near incubation temperature of 37° C.±0.5° C., and by means of a heater 42, the inside is maintained at a near incubation temperature of 37° C.±0.5° C. The passage part storage container 176, the cell suspension storage container 31 and the microtiter plates 71 are also warmed up to 37° C.±0.5° C.

Once the cell culture apparatus 100 is in its initial state, the PC 101 is used to operate the XY stage 161 and the ZY stage 166 so that the dispensation head unit 170 is positioned directly above the desired passage part 173 in the passage part storage container 176. Then, the dispensation head unit 170 is lowered so that the rear part 173a of the passage part 173 is inserted into the passage part socket 172a. In this state, electric current is passed through the electromagnetic coil 172b, generating an electromagnetic force, and thus fixing the passage part 173 to the passage part detaching joint 172.

Once the passage part 173 is fixed, the XY stage 161 and the ZY stage 166 are operated to insert the passage part 173 of the dispensation head unit 170 into the cell suspension storage container 31. Then, the syringe pump part 60 is operated so that cell suspension C is sucked through the nozzle 174 into the passage 175. After sucking, the dispensation head unit 170 is positioned directly above the opening 12 of the cell array holding chamber 110. Then, the rotary actuator 111c is driven to open the shutter 14, and the dispensation head unit 170 is lowered. That is, the passage part 173 is inserted into the cell array holding chamber 110. The PC 101 stops the lowering of the dispensation head unit 170 when the nozzle 174 is about 0.5 mm above the cell array board 2.

Next, the XY stage 161 and the ZY stage 166 are moved in minute steps, maintaining the distance between the nozzle 174 and cell array board 2 so that cell suspension C is serially discharged onto the cell array board 2 as droplet culture solutions A. After discharge is complete, the dispensation head unit 170 is withdrawn from the cell array holding chamber 110, and the shutter 14 is closed. The hold of the electromagnetic coil 172b on the passage part 173 is then released, and the passage part 173 is stored in the passage part storage container 176.

Once the droplet culture solutions A have been discharged and held onto the cell array board 2, the object lens 163 is used to observe the culture solution A droplets, and the observation images of the droplets are taken by the imaging part 165 and stored on the PC 101. The PC 101 analyzes the obtained droplet images by programming such as image processing programs to measure the diameter of the droplet. In this way, from the observation to the measurement of the diameter of culture solutions A is repeated every 10 seconds for example. If the diameter of culture solution A decreases by 10% or more than the initial state when first discharged, it is concluded that the amount of water lost due to evaporation is greater than the amount of water absorbed from the droplet mist B so that the amount of droplet mist B sprayed is increased.

The PC 101 can increase the amount of droplet mist B by increasing the applied voltage to the oscillator 24 of the ultrasonic humidifier 112. This results in an increase in the supply of droplet mist B to the cell array holding chamber 110 so that droplet culture solution A can more easily absorb the droplet mist B.

On the other hand, if the diameter of the culture solution A increases by 10% or more than the initial state when first discharged, it is concluded that the amount of water lost due to evaporation is less than the amount of water absorbed from the droplet mist B so that the amount of droplet mist B sprayed is decreased. The PC 101 can decrease the amount of droplet mist B by decreasing the applied voltage to the oscillator 24 of the ultrasonic humidifier 112.

By performing such controls mentioned above, even when the equilibrium between evaporation of the droplet culture solution A and absorption from the droplet mist B breaks down, the droplet volume of the culture solution A can be maintained at the optimal amount, that is, the amount that is first discharged. The PC 101 previously stores data regarding applied voltages for the oscillator 24 in typical temperature and humidity environments, and regarding the rate of change of the droplet diameter, to determine the applied voltage for the oscillator 24 based on this data.

Addition of drugs to the cell culture mediums held on the cell array board 2 is performed in the same manner as described above. Once the passage part 173 is fixed onto the dispensation head unit 170, the desired drug is sucked from the microtiter plate 71. Thereafter, the shutter 14 is opened to add the drug to the cell culture medium. If different drugs are to be added, an other passage part 173 is taken from the passage part storage container 176 and used.

As mentioned above, the liquid volume adjustment mechanism 130 in the cell culture apparatus 100 adjusts the liquid volume of the droplet mist B based on the detected value of the liquid volume of the culture solution A detected by the liquid volume detection mechanism 120 so that is becomes possible to maintain the liquid volume of the culture solution A at a constant volume at all times. That is, it prevents the cells from becoming dehydrated from evaporation of the culture solution A due to the supply of the droplet mist B being too small, and also prevents the culture solution A from becoming jointed with adjacent culture solutions A due to the supply of the droplet mist B being too large. Consequently, the size of the culture solution A can be maintained at its initial state optimal size, thus further decreasing the possibility of evaporation.

The liquid volume detection mechanism 120 measures the diameter of the droplet of the culture solution A and detects the liquid volume by image processing, so that the diameter of the droplet can be reliably detected and the liquid volume can be detected with high accuracy.

### EXAMPLE

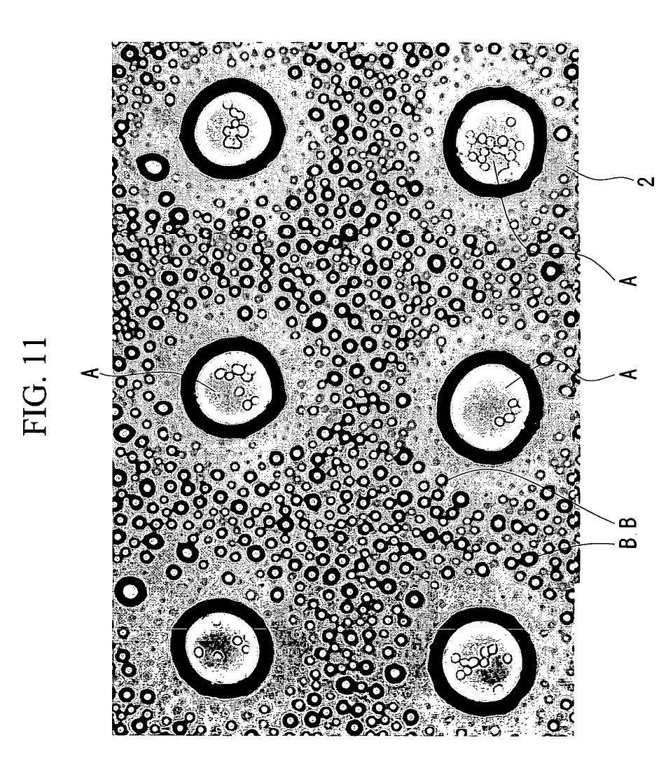

Next is a description of an example in the case where live cells were actually discharged onto the cell array board 2, with reference to FIG. 11, based on the first embodiment.

In this example, pneumocyte cells from the Chinese Hamster were used, and MEM (Minimum essential medium (Sigma Company Brand)) including 10% cow embryo serum was used as the culture solution. The cell suspension C concentration was adjusted to 2.0×10⁷/ml. The cell array board 2 was coated with fibronectin.

With the conditions stated above, cell-containing droplet culture solution A was discharged onto the cell array board 2 as shown in FIG. 11, and the presence of the droplet mist B of diameter 5 μm to 15 μm surrounding the culture solution A was confirmed. The culture solution A was held for about one hour while evaporation was prevented by the droplet mist B, and after this time it was confirmed that cellular adhesive proteins were secreted by the cells and the cells had adhered onto the cell array board 2.

The technical scope of the present invention is not limited to the above embodiments, and various modifications are possible within a scope which does not deviate from the gist of the present invention.

For example, in the first embodiment, the droplet mist was supplied irrespective of the liquid volume of the culture solution contained inside the cell array holding chamber. However the configuration may be such that the volume detection mechanism or the liquid volume adjustment mechanism of the second embodiment are provided.

Moreover, the configuration may be such that the inverted microscope in the second embodiment is added with a fluorescent cube or a phase difference ring. In this case, fluorescent observation and phase difference observation of the cells can be performed so that it becomes possible to determine the life or death of the cells and measure the activity levels of the cells. That is, it becomes possible to evaluate cell response towards drugs. Furthermore, when adding drugs to the cell culture mediums on the cell array board, the drugs can be reliably added to the cells by previously measuring the position of the cells by an inverted microscope and moving and positioning the dispensation head unit based on the measurement result.

Furthermore, regarding the cell array board, a board coated with cellular adhesive proteins on the whole surface is used. However it is not limited to this, and as with conventional methods, a board where a plurality of independent hydrophilic regions are formed inside hydrophobic regions, or a board where a plurality of independent cellular adhesive regions are formed inside cellular non-adhesive regions can also be used. If such a board is used, cell suspensions can be reliably discharged into the desired region by measuring the region to be discharged, that is the hydrophilic or cellular adhesive position, by an inverted microscope before discharging the cell suspension onto the board, and moving and positioning the dispensation head based on the measured position.

Furthermore, to prevent contamination of the cells, at the very least all parts of the inverted microscope above the stage must be covered by a clean box, and when not in use a germicidal lamp should be lit inside this clean box.

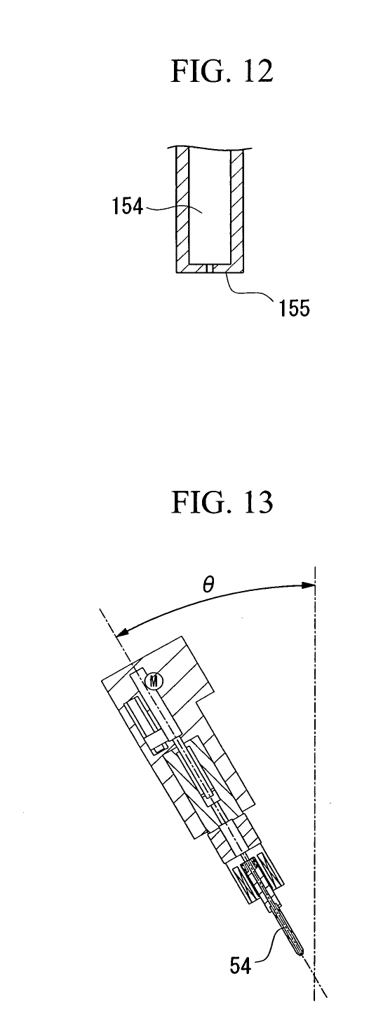

In the embodiments mentioned above, the passage inside the passage part is formed in a tapered shape, that is the diameter decreases towards the tip of the nozzle. However it is not limited to such a shape. For example, a straight passage 154 can be used as shown in FIG. 12. Particularly, when discharging high concentration cell suspensions, a straight passage 154 is preferable. With a tapered passage, cells in the cell suspension inside the passage are precipitated at the tip as time goes by, resulting in a higher cell concentration near the tip of the passage. In extreme cases, blockage of the nozzle can occur. Compared to this, in the straight passage 154, blockages are much more difficult to occur at the nozzle 155 due to the precipitation of cells. Hence as mentioned above, it is suitable for discharging high concentration cell suspensions.

As shown in FIG. 13, the passage 54 may be tilted in order to restrict the precipitation of cells inside the passage. In this case, an angle value θ of 30° or more can slow the time for blockage to occur, and an angle value θ of 60° or more can almost eliminate blockage entirely. Also, making the density of the culture solution in the cell suspension close to the density of the cells, is effective for slowing the precipitation of cells.

As described above, according to the cell culture apparatus of this invention, evaporation of the culture solution can be prevented without being a burden on the cells.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A cell culture apparatus comprising:
a board which holds cell-containing culture solution;
a holding chamber which accommodates the board in an interior space set to an incubation temperature; and
a droplet supplying device which supplies droplet mist at near incubation temperature into the interior space of the holding chamber.

2. A cell culture apparatus according to claim 1, wherein the droplet supplying device comprises:
a liquid holding container which holds a liquid; and
a droplet generating device which applies ultrasonic vibration to the liquid held in the liquid holding container to generate droplet mist.

3. A cell culture apparatus according to claim 2, wherein the droplet supplying device comprises:
an airflow generating device which generates an air flow for sending the droplet mist generated inside the liquid holding container into the holding chamber.

4. A cell culture apparatus according to claim 3, comprising a flow rate adjusting device which adjusts the flow rate of the air flow generated by the air flow generating device.

5. A cell culture apparatus according to claim 4, comprising a gas temperature adjusting device which adjusts the temperature of the gas sent into the holding chamber from the air flow generating device.

6. A cell culture apparatus according to claim 2, comprising a liquid temperature adjusting device which adjusts the temperature of the liquid held inside the liquid holding container.

7. A cell culture apparatus according to claim 1, comprising a temperature holding device which maintains the inside of the holding chamber at near incubation temperature.

8. A cell culture apparatus according to claim 7, wherein the temperature holding device comprises:
a temperature sensor arranged near the board held inside the holding chamber;
a heater which heats the board or the vicinity thereof, and
a controlling device which controls the output of the heater based on a detection value of the temperature sensor.

9. A cell culture apparatus according to claim 1 comprising a liquid discharging device which discharges liquid towards the board,
and the holding chamber comprises an opening for inserting the liquid discharging device into the holding chamber, and an opening and closing device which opens and closes the opening.

10. A cell culture apparatus according claim 9, wherein the liquid discharging device comprises a shielding device which closes a gap between the opening.

11. A cell culture apparatus according to claim 1 comprising:
a liquid volume detecting device which detects the liquid volume of the culture solution in the holding chamber; and
a liquid volume adjusting device which adjusts the volume of the droplet mist supplied by the droplet supplying device, based on the detected value of the liquid volume detecting device.

12. A cell culture apparatus according claim 11, wherein the liquid volume detecting device detects the liquid volume by image processing.

13. A cell culture apparatus according claim 12, wherein the liquid volume detecting device detects the liquid volume by measuring the droplet diameter of the culture solution.
